(19) United States
(12) Patent Application Publication
(10) Pub. No.: US 2024/0075346 A1
(43) Pub. Date: Mar. 7, 2024

(54) EXERCISE MONITORING METHOD, EXERCISE MONITORING DEVICE, AND COMPUTER-READABLE STORAGE MEDIUM

(71) Applicant: BOMDIC INC., New Taipei City (TW)

(72) Inventors: Yao Shiao, New Taipei City (TW); Shih-Chieh Wu, New Taipei City (TW)

(73) Assignee: BOMDIC INC., New Taipei City (TW)

(21) Appl. No.: 18/459,427

(22) Filed: Sep. 1, 2023

Related U.S. Application Data
(60) Provisional application No. 63/402,957, filed on Sep. 1, 2022.

(51) Int. Cl. A63B 24/00

(52) U.S. Cl. CPC A63B 24/0087; A63B 24/0062; A63B 2024/0068; A63B 2024/0096; A63B 2230/405

(57) ABSTRACT

An exercise monitoring method, an exercise monitoring device, and a computer-readable storage medium are provided. The method includes the following: obtaining an exercise course input by a user; detecting a reference respiratory pattern of the user performing the exercise course through a wearable device within a reference time interval; detecting a first respiratory pattern of the user performing the exercise course through the wearable device within a first time interval; and providing a first exercise adjustment suggestion based on a first comparison result between the first respiratory pattern and the reference respiratory pattern.

### CROSS-REFERENCE TO RELATED APPLICATION

This application claims the priority benefit of U.S. provisional application Ser. No. 63/402,957, filed on Sep. 1, 2022. The entirety of the above-mentioned patent application is hereby incorporated by reference herein and made a part of this specification.

### BACKGROUND

### Technical Field

The disclosure relates to an exercise monitoring mechanism, and in particular to an exercise monitoring method and an exercise monitoring device.

### Description of Related Art

In modern day living, many people go running to maintain physical health. Generally speaking, runners will adjust their respiratory rhythm when running to reduce the fatigue of running so that they may run for a longer distance. In addition, there are also studies indicating that rhythmic breathing may reduce the chance of physical injury.

However, as the running distance increases, the ordinary runner may be unable to maintain an appropriate respiratory rhythm due to fatigue or other reasons, which may affect the running performance. The runner may not reach the predetermined distance, or may even get injured.

Therefore, for those skilled in the art, if it is possible to design a mechanism that may help runners maintain the respiratory rhythm, the performance of the runners may be improved.

### SUMMARY

In view of this, an exercise monitoring method, an exercise monitoring device, and a computer-readable storage medium are provided in the embodiments of the disclosure to solve the technical issues.

The embodiment of the disclosure provides an exercise monitoring method for an exercise monitoring device. The method includes the following: obtaining an exercise course input by a user; detecting a reference respiratory pattern of the user performing the exercise course through a wearable device within a reference time interval; detecting a first respiratory pattern of the user performing the exercise course through the wearable device within a first time interval; providing a first exercise adjustment suggestion based on a first comparison result between the first respiratory pattern and the reference respiratory pattern.

The embodiment of the disclosure provides an exercise monitoring device including a storage circuit and a processor. The storage circuit stores a program code. The processor is coupled to the storage circuit and accesses the program code to perform the following: obtaining an exercise course input by a user; detecting a reference respiratory pattern of the user performing the exercise course through a wearable device within a reference time interval; detecting a first respiratory pattern of the user performing the exercise course through the wearable device within a first time interval; providing a first exercise adjustment suggestion based on a first comparison result between the first respiratory pattern and the reference respiratory pattern.

The embodiment of the disclosure provides a computer-readable storage medium. The computer-readable storage medium records an executable computer program, and the executable computer program is loaded by an exercise monitoring device to perform the following: obtaining an exercise course input by a user; detecting a reference respiratory pattern of the user performing the exercise course through a wearable device within a reference time interval; detecting a first respiratory pattern of the user performing the exercise course through the wearable device within a first time interval; providing a first exercise adjustment suggestion based on a first comparison result between the first respiratory pattern and the reference respiratory pattern.

### BRIEF DESCRIPTION OF THE DRAWINGS

The accompanying drawings are included for further comprehension of the disclosure, and the accompanying drawings are incorporated into the specification and constitute a part of the specification. The drawings illustrate the embodiments of the disclosure, and together with the description serve to explain the principles of the disclosure.

FIG. 1 is a schematic diagram of an exercise monitoring device according to an embodiment of the disclosure.

FIG. 2 is a flow chart of an exercise monitoring method according to an embodiment of the disclosure.

### DESCRIPTION OF THE EMBODIMENTS

Reference will now be made in detail to the exemplary embodiments. Examples of exemplary embodiments are described in the accompanying drawings. Wherever possible, the same reference symbols are used to denote the same or similar parts in the drawings and the description.

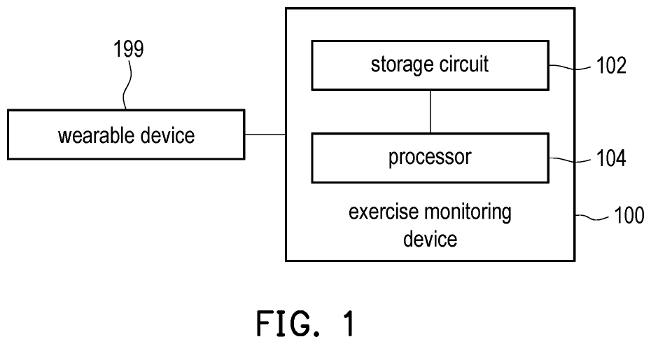

FIG. 1 is a schematic diagram of an exercise monitoring device according to an embodiment of the disclosure.

In different embodiments, an exercise monitoring device 100 may be implemented as various intelligent devices and/or computer devices, for example, but is not limited thereto. In some embodiments, the exercise monitoring device 100 may also be implemented as various wearable devices, such as various earphones, but is not limited thereto.

In FIG. 1, the exercise monitoring device 100 includes a storage circuit 102 and a processor 104. The storage circuit 102 is, for example, any type of fixed or removable random access memory (RAM), a read-only memory (ROM), a flash memory, a hard disk, other similar devices, or combinations of these devices, and may be used to record multiple program codes or modules.

The processor 104 is coupled to the storage circuit 102 and may be a general-purpose processor, a special-purpose processor, a traditional processor, a digital signal processor, multiple microprocessors, one or more microprocessors combining with a core the digital signal processor, a controller, a microcontroller, an application specific integrated circuit (ASIC), a field programmable gate array (FPGA), an integrated circuit of any other types, a state machine, an advanced RISC machine (ARM) based processor, and a similar product.

In the embodiment of the disclosure, the processor 104 may access the modules and the program codes recorded in the storage circuit 102 to implement an exercise monitoring method proposed by the disclosure, and details are described below.

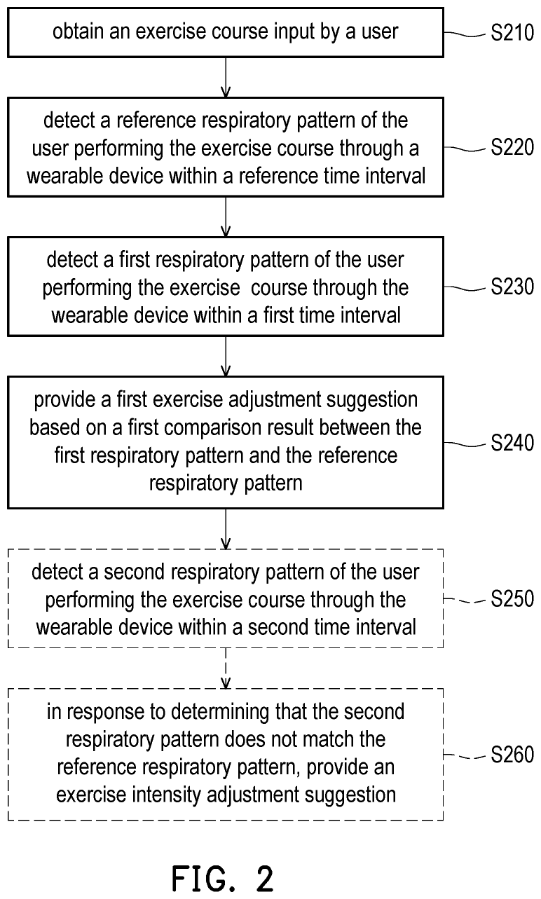

FIG. 2 is a flowchart of an exercise monitoring method according to an embodiment of the disclosure. The method of this embodiment may be executed by the exercise monitoring device 100 in FIG. 1, and details of each step in FIG. 2 are described below with reference to the components shown in FIG. 1.

In step S210, the processor 104 obtains an exercise course input by a user.

In an embodiment of the disclosure, the exercise monitoring device 100 may run a sports-related application, for example. The application may provide various exercise courses for the user to choose from, so as to input the exercise courses the user wants to perform, for example. In different embodiments, different exercise courses may have different goals of sports, for example.

For example, for running events, different exercise courses may have different goals of sports such as a target distance, a target time, a target intensity, etc. The user may, for example, select the desired exercise course according to actual needs in the application. For another example, the user may also input different goals of sports such as the target distance, the target time, the target intensity, etc. The application may plan the exercise course required by the user based on the goals of sports input by the user.

In yet a further example, for spinning or cycling events, different exercise courses may have different goals of sports such as different target riding distances, target riding times, target riding intensities (such as a resistance or watts), etc. The user may, for example, select the desired exercise course according to actual needs in the application. Similarly, the user may also input different goals of sports such as the target riding distance, the target riding time, and the target riding intensity, and the application may plan the exercise course required by the user based on the goals of sports input by the user.

In some embodiments, in response to the exercise monitoring device 100 being implemented as the wearable device such as the earphones, the user may, for example, operate the application on another electronic device (such as a smartphone) connected/paired with the exercise monitoring device 100 to select the desired exercise course. However, the disclosure is not limited thereto.

In step S220, the processor 104 detects a reference respiratory pattern of the user performing the exercise course through a wearable device 199 within a reference time interval. In an embodiment, the wearable device 199 is, for example, the earphones connected to the exercise monitoring device 100. In this case, the processor 104 may collect an in-ear signal generated by the user within the reference time interval through the earphones, and determine the reference respiratory pattern of the user based on the in-ear signal. In an embodiment, the wearable device 199 is, for example, in a form of insert earphones. A microphone component on the wearable device 199 may be used to collect a sound of an air flow when a human is breathing. A time-domain analysis and a frequency-domain analysis may be used to identify the sound of an inhalation and the sound of an exhalation in real time, thereby determining the respiratory pattern. In another embodiment, the wearable device 199 may also be any earphone that may detect the respiratory pattern of the user. In yet another embodiment, the wearable device 199 may also be any wearable device 199 that may detect the respiratory pattern of the user. Furthermore, in another embodiment, the wearable device 199 may also detect the respiratory pattern of the user in any other manner.

For example, it is assumed that the reference time interval is the first N minutes before the user starts the exercise course (N may be set to any value according to the needs of the designer). In this case, the processor 104 may collect the in-ear signal of the user during the first N minutes of the exercise course through the earphones worn on the ears of the user, and determine the respiratory pattern of the user within the N minutes accordingly as the reference respiratory pattern. In an embodiment, N may be the time of a warm-up phase of the exercise course, a period of time at a beginning of a training phase, or the time of the warm-up phase plus the period of time at the beginning of the training phase.

In another embodiment, the processor 104 may also detect the reference respiratory pattern of the user performing the exercise course through the wearable device 199 within a reference distance.

In this case, the processor 104 may collect the in-ear signal generated by the user within the reference distance through the earphones, and determine the reference respiratory pattern of the user based on the in-ear signal.

For example, it is assumed that the reference distance is first M meters before the user starts the exercise course (M may be set to any value according to the needs of the designer). In this case, the processor 104 may collect the in-ear signal of the user within the first M meters of performing the exercise course through the earphones worn on the ears of the user, and determine the respiratory pattern of the user in the first M meters accordingly as the reference respiratory pattern. In an embodiment, N may be the distance in the warm-up phase of the exercise course, the distance at the beginning of the training phase, or the distance in the warm-up phase plus the distance at the beginning of the training phase.

Furthermore, when the user starts the exercise course (such as the first N minutes and/or M meters), the relatively stable and required respiratory pattern is generally maintained. Therefore, the reference respiratory pattern determined at this time may be closer to the preferred/expertise/suitable respiratory pattern of the user.

In some embodiments, when the exercise monitoring device 100 is implemented as the earphones worn by the user, the exercise monitoring device 100 may detect the in-ear signal of the user within the reference distance through a sensor of the exercise monitoring device 100, and determine the reference respiratory pattern accordingly. However, the disclosure is not limited thereto.

In the embodiment of the disclosure, the reference respiratory pattern includes a reference respiratory rhythm (such as inhaling-inhaling-exhaling, inhaling-exhaling-exhaling, inhaling-inhaling-exhaling-exhaling, etc.) and/or a reference respiratory frequency (such as the time required for completing the reference respiratory rhythm once and/or the number of times the reference respiratory rhythm is completed within a fixed time) of the user within the reference time interval (and/or the reference distance). However, the disclosure is not limited thereto.

In step S230, the processor 104 detects a first respiratory pattern of the user performing the exercise course through the wearable device 199 within a first time interval. In the embodiment of the disclosure, the first time interval is, for example, a certain time interval after the reference time interval, and the designer and/or the user may set a start time point of the first time interval according to the actual needs. However, the disclosure is not limited thereto.

In different embodiments, the start time point of the first time interval may be determined by the designer and/or the user based on an experiment and/or an experience. For example, assuming that some experiments indicate that an ordinary runner may have disorders in the respiratory rhythm after K1 minutes from the start of running, the start time point of the first time interval may be designed to be close to K1 minutes. In another example, assuming that the user knows that the disorders in the respiratory rhythm may occur after K2 minutes from the start of running, the start time point of the first time interval may be designed to be close to K2 minutes. However, the disclosure is not limited thereto.

In another embodiment, the start time point of the first time interval may also be determined according to a content of the exercise course. For example, the start time point of the first time interval may be set to the time point when the user completes a certain percentage of the exercise course. Assuming that the specific percentage is 50%, the processor 104 may, for example, use the time point at which the user completes 50% of the target distance of the exercise course as the start time point of the first time interval. However, the disclosure is not limited thereto.

In yet another embodiment, the start time point of the first time interval may also immediately follow the reference time interval to continuously observe a breathing status of the user.

In the embodiment of the disclosure, a length of the first time interval may also be determined by the designer/user according to actual needs. In an embodiment, the length of the first time interval may be set to a unit time or a fixed value based on the exercise course. The unit time is at least long enough to detect that the user has completed several breaths and determine the respiratory pattern of the user. However, the disclosure is not limited thereto.

In an embodiment, the first respiratory pattern includes, for example, a first respiratory rhythm (such as inhaling-inhaling-exhaling, inhaling-exhaling-exhaling, inhaling-inhaling-exhaling-exhaling, etc.) and/or a first respiratory frequency (such as the time required for completing the first respiratory rhythm once and/or the number of times the first respiratory rhythm is completed within the fixed time) of the user within the first time interval. However, the disclosure is not limited thereto.

In step S240, the processor 104 provides a first exercise adjustment suggestion based on a first comparison result between the first respiratory pattern and the reference respiratory pattern.

In the first embodiment, in response to determining that the first comparison result indicates that the first respiratory rhythm does not match the reference respiratory rhythm, the processor 104 may provide a respiratory rhythm adjustment suggestion as the first exercise adjustment suggestion.

In the second embodiment, in response to determining that the first comparison result indicates that the first respiratory frequency does not match the reference respiratory frequency, the processor 104 may provide a respiratory frequency adjustment suggestion as the first exercise adjustment suggestion. For example, in response to determining that the first respiratory frequency is higher than the reference respiratory frequency, it is indicated that the breaths of the user become more rapid. In this case, the processor 104 may provide a respiratory frequency reduction suggestion as a respiratory frequency adjustment suggestion.

In the third embodiment, in response to determining that the first comparison result indicates that the first respiratory rhythm does not match the reference respiratory rhythm, and the first respiratory frequency does not match the reference respiratory frequency, the processor 104 may simultaneously provide the respiratory rhythm adjustment suggestion and the respiratory frequency adjustment suggestion as the first exercise adjustment suggestion. However, the disclosure is not limited thereto.

In an embodiment, the processor 104 may play a specific sound/voice through the earphones to present the first exercise adjustment suggestion. For example, when the processor 104 determines that the first respiratory rhythm (for example, inhaling-inhaling-exhaling-exhaling) of the user in the first time interval is different from the reference respiratory rhythm (for example, inhaling-inhaling-exhaling) in the reference time interval, the processor 104, for example, may control the earphones to play corresponding voice prompts to remind the user to resume the reference respiratory rhythm.

In another example, when the processor 104 determines that the first respiratory frequency of the user in the first time interval (for example, about 45 times of the first respiratory rhythm per minute) is different from the reference respiratory frequency in the reference time interval (for example, about 15 times of the first respiratory rhythm per minute), the processor 104 may, for example, control the earphones to play the corresponding voice prompts to remind the user to resume the reference respiratory frequency. However, the disclosure is not limited thereto.

In another embodiment, the processor 104 may also control a body of the exercise monitoring device 100, the earphones, and/or other wearable devices 199 to generate vibrations in a specific manner as the first exercise adjustment suggestion. However, the disclosure is not limited thereto.

In this way, the user may adjust the respiratory rhythm and/or the respiratory frequency according to the respiratory rhythm adjustment suggestion and/or the respiratory frequency adjustment suggestion, thereby continuing the exercise course in a proper condition and reducing a risk of injury.

In an embodiment, after step S240, the processor 104 may further perform step S250 to detect a second respiratory pattern of the user performing the exercise course through the wearable device 199 within a second time interval.

In the embodiment of the disclosure, the second time interval is, for example, the first time interval and/or the time interval after the first exercise adjustment suggestions is provided. The designer and/or the user may set the start time point of the second time interval according to actual needs. However, the disclosure it is not limited thereto.

For example, assuming that the user may need a period of time, such as several minutes, to complete a corresponding adjustment after receiving the first exercise adjustment suggestion, the start time point of the second time interval may be set at the several minutes after the processor 104 provides the first exercise adjustment suggestion. In another embodiment, a time difference between the start time point of the second time interval and the time point when the processor 104 provides the first exercise adjustment suggestion may also be a fixed value. However, the disclosure is not limited thereto.

In the embodiment of the disclosure, the second respiratory pattern includes a second respiratory frequency (such as the time required to complete current respiratory rhythm once and/or the times of the current respiratory rhythm complete within the fixed time) of the user within the second time interval. Generally, ideally, after the user receives the first exercise adjustment suggestion, the current respiratory rhythm may be adjusted to present the reference respiratory rhythm. In other embodiments, the processor 104 may also directly use the respiratory rhythm measured in the second time interval as the current respiratory rhythm under consideration. However, the disclosure is not limited thereto.

Next, in step S260, in response to determining that the second respiratory pattern does not match the reference respiratory pattern, the processor 104 provides an exercise intensity adjustment suggestion.

In an embodiment, in response to determining that the second respiratory frequency is higher than the reference respiratory frequency, the user may not be able to smoothly reduce the respiratory frequency according to the previously received first exercise adjustment suggestion. In other words, the user may be performing the exercise course at an extremely high intensity. In this case, the processor 104 may, for example, provide an exercise intensity reduction suggestion as the exercise intensity adjustment suggestion.

In the embodiment of the disclosure, the processor 104 may also use the sound/voice/vibration and other methods as the exercise intensity adjustment suggestion. For example, when the processor 104 determines that the second respiratory frequency of the user in the second time interval is higher than the reference respiratory frequency, the processor 104 may, for example, control the earphones to play the corresponding voice prompt to remind the user to reduce the current exercise intensity (for example, running or cycling at a lower speed). However, the disclosure is not limited thereto.

In addition, the disclosure further provides a computer-readable storage medium for performing the exercise monitoring method. The computer-readable storage medium consists of multiple program instructions (for example, setup program instructions and deployment program instructions) implemented therein. The program instructions may be loaded into the exercise monitoring device 100 and executed by the exercise monitoring device 100 to perform the exercise monitoring method and the functions of the exercise monitoring device 100.

In summary, the technical solution proposed by the embodiment of the disclosure may provide the respiratory rhythm adjustment suggestion and/or the respiratory frequency adjustment suggestion when it is determined that the reference respiratory pattern measured within the reference time interval does not match the first respiratory pattern measured within the first time interval.

Furthermore, the embodiment of the disclosure may also remind the user to reduce the exercise intensity by providing the exercise intensity adjustment suggestion when the user fails to smoothly reduce the respiratory frequency according to the first exercise adjustment suggestion. In this way, the user may adjust the respiratory rhythm and/or the respiratory frequency according to the first exercise adjustment and/or the exercise intensity adjustment suggestion, thereby continuing the exercise course in the proper condition to complete the exercise course and reducing the risk of injury.

Finally, it should be noted that the above embodiments are only used to illustrate, but not to limit, the technical solutions of the disclosure. Although the disclosure has been described in detail with reference to the above embodiments, persons skilled in the art should understand that the technical solutions described in the above embodiments may still be modified or some or all of the technical features thereof may be equivalently replaced. However, the modifications or replacements do not cause the essence of the corresponding technical solutions to deviate from the scope of the technical solutions of the embodiments of the disclosure.

What is claimed is:

1. An exercise monitoring method for an exercise monitoring device, comprising:
obtaining an exercise course input by a user;
detecting a reference respiratory pattern of the user performing the exercise course through a wearable device within a reference time interval;
detecting a first respiratory pattern of the user performing the exercise course through the wearable device within a first time interval; and
providing a first exercise adjustment suggestion based on a first comparison result between the first respiratory pattern and the reference respiratory pattern.

2. The method according to claim 1, wherein the reference respiratory pattern comprises a reference respiratory rhythm of the user within the reference time interval, and the first respiratory pattern comprises a first respiratory rhythm of the user within the first time interval; and
wherein providing the first exercise adjustment suggestion based on the first comparison result between the first respiratory pattern and the reference respiratory pattern comprises:
providing a respiratory rhythm adjustment suggestion as the first exercise adjustment suggestion in response to determining that the first respiratory rhythm does not match the reference respiratory rhythm.

3. The method according to claim 1, wherein the reference respiratory pattern comprises a reference respiratory frequency of the user within the reference time interval, the first respiratory pattern comprises a first respiratory frequency of the user within the first time interval; and
wherein providing the first exercise adjustment suggestion based on the first comparison result between the first respiratory pattern and the reference respiratory pattern comprises:
providing a respiratory frequency adjustment suggestion as the first exercise adjustment suggestion in response to determining that the first respiratory frequency does not match the reference respiratory frequency.

4. The method according to claim 3, wherein providing the respiratory frequency adjustment suggestion as the first exercise adjustment suggestion comprises:
providing a respiratory frequency reduction suggestion as the respiratory frequency adjustment suggestion in response to determining that the first respiratory frequency is higher than the reference respiratory frequency.

5. The method according to claim 1, wherein after providing the first exercise adjustment suggestion based on the first comparison result between the first respiratory pattern and the reference respiratory pattern, the method further comprises:
detecting a second respiratory pattern of the user performing the exercise course through the wearable device within a second time interval; and
providing an exercise intensity adjustment suggestion in response to determining that the second respiratory pattern does not match the reference respiratory pattern.

6. The method according to claim 5, wherein the reference respiratory pattern comprises a reference respiratory frequency of the user within the reference time interval, the second respiratory pattern comprises a second respiratory frequency of the user within the second time interval, and providing the exercise intensity adjustment suggestion comprises:
providing an exercise intensity reduction suggestion as the exercise intensity adjustment suggestion in response to determining that the second respiratory frequency is higher than the reference respiratory frequency.

7. The method according to claim 1, wherein the exercise course comprises a target distance.

8. The method according to claim 1, wherein the wearable device is earphones, and detecting the reference respiratory pattern within the reference time interval of the user performing the exercise course through the wearable device comprises:
collecting an in-ear signal generated by the user within the reference time interval through the earphones; and
determining the reference respiratory pattern based on the in-ear signal.

9. An exercise monitoring device, comprising:
a storage circuit, storing a program code; and
a processor, coupled to the storage circuit, accessing the program code to perform:
obtaining an exercise course input by a user;
detecting a reference respiratory pattern of the user performing the exercise course through a wearable device within a reference time interval;
detecting a first respiratory pattern of the user performing the exercise course through the wearable device within a first time interval; and
providing a first exercise adjustment suggestion based on a first comparison result between the first respiratory pattern and the reference respiratory pattern.

10. A computer-readable storage medium, wherein the storage computer-readable medium records an executable computer program, and the executable computer program is loaded by an exercise monitoring device to perform:
obtaining an exercise course input by a user;
detecting a reference respiratory pattern of the user performing the exercise course through a wearable device within a reference time interval;
detecting a first respiratory pattern of the user performing the exercise course through the wearable device within a first time interval; and
providing a first exercise adjustment suggestion based on a first comparison result between the first respiratory pattern and the reference respiratory pattern.
